(19) United States
(12) Patent Application Publication
(10) Pub. No.: US 2005/0226922 A1
(43) Pub. Date: Oct. 13, 2005

(54) Apparatus and method for transdermal delivery of fentanyl-based agents

(76) Inventors: Mahmoud Ameri, Fremont, CA (US); Michel J.N. Cormier, Mountain View, CA (US); Yuh-Fun Maa, Millbrae, CA (US); Peter Daddona, Menlo Park, CA (US)

Correspondence Address: Ralph C. Francis;Francis Law Group, 1942 Embarcadero, Oakland, CA, 94606, US

(21) Appl. No.: 11/084,636

(22) Filed: Mar. 18, 2005

Related U.S. Application Data
(60) Provisional application No. 60/561,949, filed on Apr. 13, 2004.

(51) Int. Cl. A61K 31/445; A61K 9/70

(52) U.S. Cl. 424/449; 514/317

(57) ABSTRACT

An apparatus and method for transdermally delivering a biologically active agent comprising a delivery system having a microprojection member (or system) that includes a plurality of microprojections (or array thereof) that are adapted to pierce through the stratum corneum into the underlying epidermis layer, or epidermis and dermis layers. In one embodiment, the fentanyl-based agent is contained in a biocompatible coating that is applied to the microprojection member. In a further embodiment, the delivery system includes a gel pack having a fentanyl-based agent-containing hydrogel formulation that is disposed on the microprojection member after application to the skin of a patient. In an alternative embodiment, the fentanyl-based agent is contained in both the coating and the hydrogel formulation.

### CROSS-REFERENCE TO RELATED APPLICATIONS

This application claims the benefit of U.S. Provisional Application No. 60/561,949, filed Apr. 13, 2004.

### FIELD OF THE PRESENT INVENTION

The present invention relates generally to transdermal agent delivery systems and methods. More particularly, the invention relates to an apparatus and method for transdermal delivery of fentanyl-based agents.

### BACKGROUND OF THE INVENTION

Active agents (or drugs) are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and devices developed to mechanically penetrate or disrupt the outermost skin layers thereby creating pathways into the skin in order to enhance the amount of agent being transdermally delivered. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, U.S. Pat. No. Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is possible to have the active agent that is to be delivered coated on the microprojections instead of contained in a physical reservoir. This eliminates the necessity of a separate physical reservoir and developing an agent formulation or composition specifically for the reservoir.

Fentanyl and its salts (i.e., fentanyl-based agents) are typically administered in the management of pain in patients who require continuous opioid analgesia for pain that cannot be managed by lesser means, such as acetaminophen-opioid combinations, non-steroidal analgesics, or PRN dosing with short-acting opioids and for management of breakthrough pain in patients with malignancies who are already receiving and who are tolerant to opioid therapy for their underlying persistent cancer pain. At present, fentanyl is only administered by intravenous, passive transdermal and oral transmucosal routes. The provision of a transdermal administration by microprojection of a fentanyl-based agent would have a faster onset of action and higher drug utilization than the prior art transdermal and oral transmucosal routes.

It would therefore be desirable to provide an agent delivery system that facilitates transdermal administration of fentanyl-based agents.

It is therefore an object of the present invention to provide a transdermal agent delivery apparatus and method that provides transdermal delivery of a fentanyl-based agent to a patient.

It is another object of the invention to provide a fentanyl-based agent formulation for transdermal delivery to a patient.

It is another object of the present invention to provide a transdermal agent delivery apparatus and method that includes microprojections coated with a biocompatible coating that includes at least one biologically active agent, preferably, a fentanyl-based agent.

It is yet another object of the present invention to provide a transdermal agent delivery apparatus and method that includes a gel pack adapted to receive a hydrogel formulation that contains a fentanyl-based agent.

### SUMMARY OF THE INVENTION

In accordance with the above objects and those that will be mentioned and will become apparent below, the apparatus and method for transdermally delivering a fentanyl-based agent in accordance with this invention generally comprises a delivery system having a microprojection member (or system) that includes a plurality of microprojections (or array thereof) that are adapted to pierce through the stratum corneum into the underlying epidermis layer, or epidermis and dermis layers and an agent formulation containing the fentanyl-based agent that is adapted for transdermal delivery.

In a preferred embodiment, the fentanyl-based agent is selected from the group consisting of fentanyl base, fentanyl salts, including chloride and citrate, alpha-methyl fentanyl, 3-methyl fentanyl, 4-methyl fentanyl, and other simple derivatives of fentanyl and closely related molecules, including without limitation, remifentanyl, sufentanyl, alfentanyl, lofentanyl and carfentanyl.

Suitable fentanyl salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

In one embodiment of the invention, the fentanyl-based agent comprises in the range of approximately 1-60 wt. % of the coating formulation, preferably, in the range of approximately 5-30 wt. % of the coating formulation.

The counterion forming the fentanyl salt is present in amounts necessary to neutralize the positive charge present on the fentanyl based agent at the pH of the formulation. Excess of counterion (as the free acid or as a salt) can be added to the drug in order to control pH and to provide adequate buffering capacity. In the case of counterions bearing more than one negative charge, fentanyl based agent can be added in excess of the acid. For example, the citrate salt of fentanyl can be the monocitrate or the hemicitrate.

In one embodiment, the microprojection member includes a biocompatible coating on the microprojection, wherein the coating is formed from the agent formulation.

The agent formulations applied to the microprojection member to form solid biocompatible coatings can comprise aqueous and non-aqueous formulations having at least one fentanyl-based agent, which can be dissolved within a biocompatible carrier or suspended within the carrier.

In one embodiment of the invention, the coating formulation includes at least one buffer. Examples of such buffers include ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

Preferably, the pH of the coating formulation is below approximately pH 6. More preferably, the pH of the coating formulation is in the range of approximately pH 2-6. Even more preferably, the pH of the coating formulation is in the range of approximately pH 2-5.5.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, Triton X-100, Triton X-305, Brij 35, polysorbates such as Tween 20 and Tween 80, other sorbitan derivatives, such as sorbitan laurate, and alkoxylated alcohols, such as laureth-4.

In one embodiment of the invention, the concentration of the surfactant is in the range of approximately 0.01-20 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hyroxyethyl starch, dextran, poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 1-30 wt. %, more preferably, in the range of approximately 1-20 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, a polysaccharide or a reducing sugar. Suitable non-reducing sugars for use in the methods and compositions of the invention include, for example, sucrose, trehalose, stachyose, or raffinose. Suitable polysaccharides for use in the methods and compositions of the invention include, for example, dextran, soluble starch, dextrin, and inulin. Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent, which can comprise alpha-cyclodextrin, beta-cyclodextrin, gamma-cyclodextrin, glucosyl-alpha-cyclodextrin, maltosyl-alpha-cyclodextrin, glucosyl-beta-cyclodextrin, maltosyl-beta-cyclodextrin, hydroxypropyl beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, and sulfobutylether-gamma-cyclodextrin. Most preferred solubilising/complexing agents are beta-cyclodextrin, hydroxypropyl beta-cyclodextrin, 2-hydroxypropyl-beta-cyclodextrin and sulfobutylether7 beta-cyclodextrin.

The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt. % to 50 wt. % of the coating formulation.

In yet another embodiment of the invention, the coating formulation includes a suspension agent, which can form a homogenous mixture with the fentanyl-based agent. Suitable suspension agents include, without limitation, polyethylene glycol (PEG) and polyvinylpyrrolidine (PVP). A currently preferred suspension agent is PVP (50 kDa).

Preferably, the coating formulations have a viscosity less than approximately 500 centipoise and greater than 3 centipose.

In one embodiment of the invention, the thickness of the biocompatible coating is less than 25 microns, more preferably, less than 10 microns, as measured from the microprojection surface.

In one embodiment of the invention, the microprojection member has a microprojection density of at least approximately 10 microprojections/cm², preferably, greater than approximately 100 microprojections/cm², and more preferably, in the range of approximately 200-3000 microprojections/cm². Further, each of the microprojections preferably has a length in the range of approximately 50-145 microns, and more preferably, in the range of approximately 70-140 microns.

In one embodiment, the microprojection member is constructed out of stainless steel, titanium, nickel titanium alloys, or similar biocompatible materials, such as polymeric materials.

In another embodiment, the microprojection member is constructed out of a non-conductive material, such as a polymer. Alternatively, the microprojection member can be coated with a non-conductive material, such as Parylene®, or a hydrophobic material, such as Teflon®, silicon or other low energy material.

In a further embodiment of the invention, the delivery system includes a gel pack, the gel pack being adapted to receive a hydrogel formulation.

Preferably, the fentanyl-based agent comprises in the range of approximately 0.1-10 wt. % of the hydrogel formulation.

Preferably, the pH of the hydrogel formulation is below approximately pH 6. More preferably, the pH of the hydrogel formulation is in the range of approximately pH 2-6. Even more preferably the pH of the hydrogel formulation is in the range of approximately pH 2-5.5.

In one embodiment of the invention, the hydrogel formulation includes at least one of the aforementioned buffers.

The hydrogel formulation(s) contained in the gel pack preferably comprise water-based hydrogels having macromolecular polymeric networks.

In a preferred embodiment of the invention, the polymer network comprises, without limitation, hyroxyethyl starch, dextran, hydroxyethylcellulose (HEC), hydroxypropylmethylcellulose (HPMC), hydroxypropycellulose (HPC), methylcellulose (MC), hydroxyethyl-methylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

The hydrogel formulation preferably includes at least one surfactant, which can be zwitterionic, amphoteric, cationic, anionic, or nonionic.

In one embodiment of the invention, the surfactant comprises sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols such as laureth-4.

In another embodiment, the hydrogel formulation includes polymeric materials or polymers having amphiphilic properties, which can comprise, without limitation, cellulose derivatives, such as hydroxyethylcellulose (HEC), hydroxypropyl-methylcellulose (HPMC), hydroxypropycellulose (HPC), methylcellulose (MC), hydroxyethylmethylcellulose (HEMC), or ethylhydroxyethylcellulose (EHEC), as well as pluronics.

In a further embodiment of the invention, the hydrogel formulation includes a solubilising/complexing agent, which can comprise alpha-cyclodextrin, beta-cyclodextrin, gamma-cyclodextrin, glucosyl-alpha-cyclodextrin, maltosyl-alpha-cyclodextrin, glucosyl-beta-cyclodextrin, maltosyl-beta-cyclodextrin, hydroxypropyl-beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, and sulfobutylether-gamma-cyclodextrin. most preferred are beta-cyclodextrin, hydroxypropyl-beta-cyclodextrin, 2-hydroxypropyl-beta-cyclodextrin and sulfobutylether7 beta-cyclodextrin.

In another embodiment of the invention, the hydrogel formulation includes at least one non-aqueous solvent, such as ethanol, isopropanol, methanol, propanol, butanol, propylene glycol, dimethyl sulphoxide and polyethylene glycol 400. Preferably, the non-aqueous solvent is present in the range of approximately 1 wt. % to 50 wt. % of the hydrogel formulation.

In a further embodiment of the invention, the hydrogel formulation contains at least one pathway patency modulator, which can comprise, without limitation, osmotic agents (e.g., sodium chloride), zwitterionic compounds (e.g., amino acids), and anti-inflammatory agents, such as betamethoasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinate sodium salt, paramethasone disodium phosphate and prednisolone 21-succinate sodium salt, and anticoagulants, such as citric acid, citrate salts (e.g., sodium citrate), dextrin sulfate sodium, and EDTA.

In yet another embodiment of the invention, the hydrogel formulation includes at least one vasoconstrictor, which can comprise, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropolamine, propylhexedrine, pseudoephedrine, tetrahydrozoline, tramazoline, tuaminoheptane, tymazoline, vasopressin and xylometazoline, and the mixtures thereof.

In at least one additional embodiment of the invention, the hydrogel formulation contains at least one fentanyl-based agent.

In accordance with yet another embodiment of the invention, the delivery system for delivering a fentanyl-based agent includes (i) a gel pack containing a hydrogel formulation and (ii) a microprojection member having top and bottom surfaces, a plurality of openings that extend through the microprojection member and a plurality of stratum corneum-piercing microprotrusions that project from the bottom surface of the microprojection member, the microprojection member including a solid film having at least one fentanyl-based agent. In one embodiment, the solid film is disposed proximate the top surface of the microprojection member. In another embodiment, the solid film is disposed proximate the bottom surface of the microprojection member.

In a preferred embodiment, the hydrogel formulation is devoid of a fentanyl-based agent.

In one embodiment, the solid film is made by casting a liquid formulation consisting of the fentanyl-based agent, a polymeric material, such as hyroxyethyl starch, dextran, hydroxyethylcellulose (HEC), hydroxypropylmethylcellulose (HPMC), hydroxypropycellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone), or pluronics, a plasticising agent, such as glycerol, propylene glycol, or polyethylene glycol, a surfactant, such as tween 20 or tween 80, and a volatile solvent, such as water, isopropanol, methanol or ethanol.

In one embodiment, the liquid formulation used to produce the solid film comprises: 0.1-10 wt. % fentanyl-based agent, 5-40 wt. % polymer, 5-40 wt. % plasticiser, 0-2 wt. % surfactant, and the balance of volatile solvent.

Preferably, the fentanyl-based agent is present in the liquid formulation used to produce the solid film at a concentration in the range of approximately 0.1-10 wt. %.

Preferably, the pH of the liquid formulation used to produce the solid film is below about 6. More preferably, the pH of the formulation used to produce the solid film is in the range of approximately 2-6. Even more preferably, the pH of the liquid formulation used to produce the solid film is in the range of approximately 2-5.5.

In one embodiment of the invention, the liquid formulation used to produce the solid film includes at least one buffer. Examples of such buffers include ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

In another embodiment, the liquid formulation used to produce the solid film includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose. Suitable polysaccharides for use in the methods and compositions of the invention include, for example, dextran, soluble starch, dextrin, and inulin. Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

The liquid formulation used to produce the solid film preferably includes at least one surfactant, which can be zwitterionic, amphoteric, cationic, anionic, or nonionic.

In another embodiment of the invention, the surfactant comprises sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols such as laureth-4.

In a further embodiment of the invention, the liquid formulation used to produce the solid film includes a solubilising/complexing agent, which can comprise alpha-cyclodextrin, beta-cyclodextrin, gamma-cyclodextrin, glucosyl-alpha-cyclodextrin, maltosyl-alpha-cyclodextrin, glucosyl-beta-cyclodextrin, maltosyl-beta-cyclodextrin, hydroxypropyl-beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, and sulfobutylether-gamma-cyclodextrin. Most preferred are beta-cyclodextrin, hydroxypropyl-beta-cyclodextrin, 2-hydroxypropyl-beta-cyclodextrin and sulfobutylether7 beta-cyclodextrin.

In a further embodiment of the invention, the liquid formulation used to produce the solid film contains at least one pathway patency modulator, which can comprise, without limitation, osmotic agents (e.g., sodium chloride), zwitterionic compounds (e.g., amino acids), and anti-inflammatory agents, such as betamethoasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinate sodium salt, paramethasone disodium phosphate and prednisolone 21-succinate sodium salt, and anticoagulants, such as citric acid, citrate salts (e.g., sodium citrate), dextrin sulfate sodium, and EDTA.

In yet another embodiment of the invention, the liquid formulation used to produce the solid film includes at least one vasoconstrictor, which can comprise, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropolamine, propylhexedrine, pseudoephedrine, tetrahydrozoline, tramazoline, tuaminoheptane, tymazoline, vasopressin and xylometazoline, and the mixtures thereof.

In accordance with one embodiment of the invention, the method for delivering a fentanyl-based agent contained in the biocompatible coating on the microprojection member includes the following steps: the coated microprojection member is initially applied to the patient's skin via an actuator, wherein the microprojections pierce the stratum corneum. The coated microprojection member is preferably left on the skin for a period lasting from 5 seconds to 24 hours. Following the desired wearing time, the microprojection member is removed.

In accordance with a further embodiment of the invention, the method for delivering a fentanyl-based agent contained in a solid film disposed proximate to (or on) a microprojection member includes the following steps: the microprojection member 30 is initially applied to the patient's skin via an actuator, wherein the microprojections 34 pierce the stratum corneum. The microprojection member 30 is preferably left on the skin for a period lasting from 5 seconds to 24 hours. Following the desired wearing time, the microprojection member 30 is removed.

In a further aspect of the noted embodiment, the fentanyl-based agent is contained in a solid film and the hydrogel formulation is devoid of a biologically active agent and, hence, is merely a hydration mechanism.

In a further embodiment of the invention, the microprojection member is applied to the patient's skin, a gel pack having a fentanyl-based agent-containing hydrogel formulation is then placed on top of the applied microprojection member, wherein the hydrogel formulation migrates into and through the microslits in the stratum corneum produced by the microprojections. The microprojection member-gel pack assembly is preferably left on the skin for a period lasting from 5 minutes to 7 days. Following the desired wearing time, the microprojection member and gel pack are removed.

In another embodiment of the invention, the microprojection device is applied to the patient's skin and immediately removed. The gel pack having a fentanyl based agent-containing hydrogel formulation is then placed on top of the pretreated skin, wherein the hydrogel formulation migrates into and through the microslits in the stratum corneum produced by the microprojections. Preferably, the gel pack is left on the skin for a period lasting from 5 minutes to 7 days. Following the desired wearing time, the gel pack is removed.

In yet another embodiment of the invention, the microprojection member having a fentanyl-based agent-containing biocompatible coating is applied to the patient's skin, a gel pack having a fentanyl-based agent-containing hydrogel formulation is then placed on top of the applied microprojection member, wherein the hydrogel formulation and migrates into and through the microslits in the stratum corneum produced by the microprojections. The microprojection member-gel pack assembly is preferably left on the skin for a period lasting 1-6 hours, more preferably, 2-4 hours. Following the desired wearing time, the microprojection member and gel pack are removed.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection member, according to the invention;

FIG. 2 is a perspective view of the microprojection member shown in FIG. 1 having a coating deposited on the microprojections;

FIG. 3 is a side sectional view of a microprojection member having an adhesive backing, according to the invention;

FIG. 4 is an exploded perspective view of one embodiment of a gel pack of a microprojection system, according to the invention;

FIG. 5 is an exploded perspective view of one embodiment of a microprojection member of a microprojection system, according to the invention;

FIG. 6 is a perspective view of one embodiment of a microprojection assembly comprising the gel pack shown in FIG. 4 and the microprojection member shown in FIG. 5;

FIG. 7 is a side sectional view of a retainer having a microprojection member disposed therein, according to the invention;

FIG. 8 is a perspective view of the retainer shown in FIG. 7;

FIG. 9 is an exploded perspective view of an applicator and retainer, according to the invention;

FIG. 10 is a graphical illustration showing the charge profile for a fentanyl-based agent; and

FIG. 11 is a graphical illustration showing the mole ratios of a net-charged species of a fentanyl-based agent.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The terms “transdermal” and “intracutaneous”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the fentanyl-based agent is delivered, before and during transdermal flux of the fentanyl-based agent, during transdermal flux of the fentanyl-based agent, during and after transdermal flux of the fentanyl-based agent, and/or after transdermal flux of the fentanyl-based agent. Additionally, two or more fentanyl-based agents may be formulated in the coatings and/or hydrogel formulation, resulting in co-delivery of the fentanyl-based agents.

The term “fentanyl-based agent”, as used herein, includes, without limitation, fentanyl base, fentanyl salts, simple derivatives of fentanyl and closely related molecules. Examples of pharmaceutically acceptable fentanyl salts include, without limitation, acetate, propionate, butyrate, pentanoate, hexanoate, heptanoate, levulinate, chloride, bromide, citrate, succinate, maleate, glycolate gluconate, glucuronate, 3-hydroxyisobutrate, 2-hydroxyisobutyrate, lactate, malate, pyruvate, fumarate, tartarate, tartronate, nitrte, phosphate, benzene sulfonate, methane sulfonate, sulfate, sulfonate, tricarballylicate, malonate, adipate, citraconate, glutarate, itaconate, mesaconate, citramalate, dimethylolpropionate, tiglicate, glycerate, methacrylate, isocrotonate, β-hydroxybutyrate, crotonate, angelate, hydracrylate, ascorbate, aspartate, glutamate.

Examples of simple fentanyl derivatives include, without limitation, alpha-methyl fentanyl, 3-methyl fentanyl, and methyl fentanyl.

Closely related molecules include, without limitation, remifentanyl, sufentanyl, alfentanyl, lofentanyl, and carfentanyl.

The noted fentanyl-based agents can also be in various forms, such as free bases, acids, charged or uncharged molecules, components of molecular complexes or nonirritating, pharmacologically acceptable salts.

It is to be understood that more than one fentanyl-based agent can be incorporated into the agent source, reservoirs, and/or coatings of this invention, and that the use of the term “fentanyl-based agent” in no way excludes the use of two or more such active agents or drugs.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

In one embodiment of the invention, the piercing elements have a projection length less than 1000 microns. Preferably, the piercing elements have a projection length of less than 500 microns, more preferably, less than 250 microns.

In a further embodiment adapted to minimize bleeding and irritation, the microprojections preferably have a projection length less than 145 microns, more preferably, in the range of approximately 50-145 microns, and even more preferably, in the range of approximately 70-140 microns.

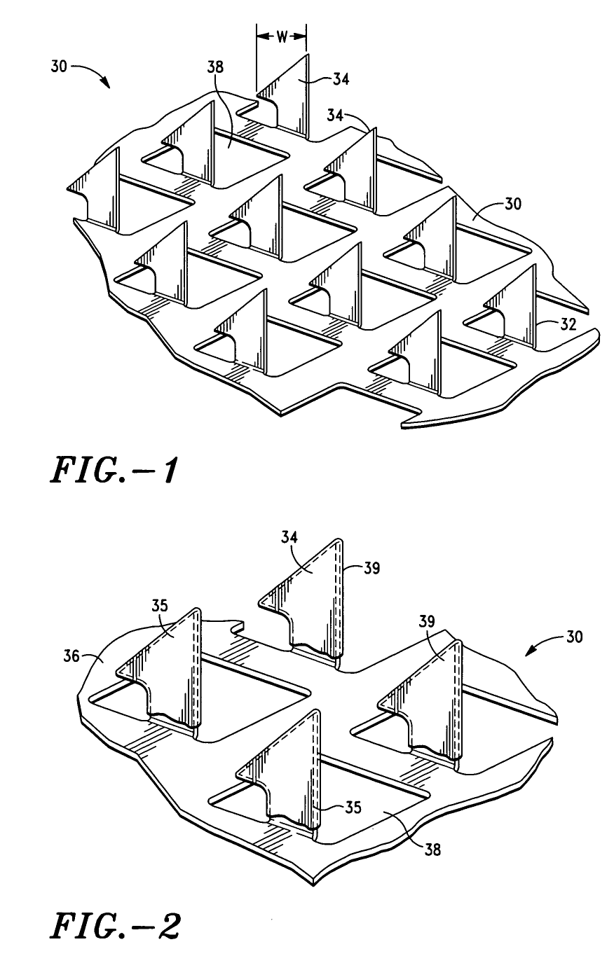

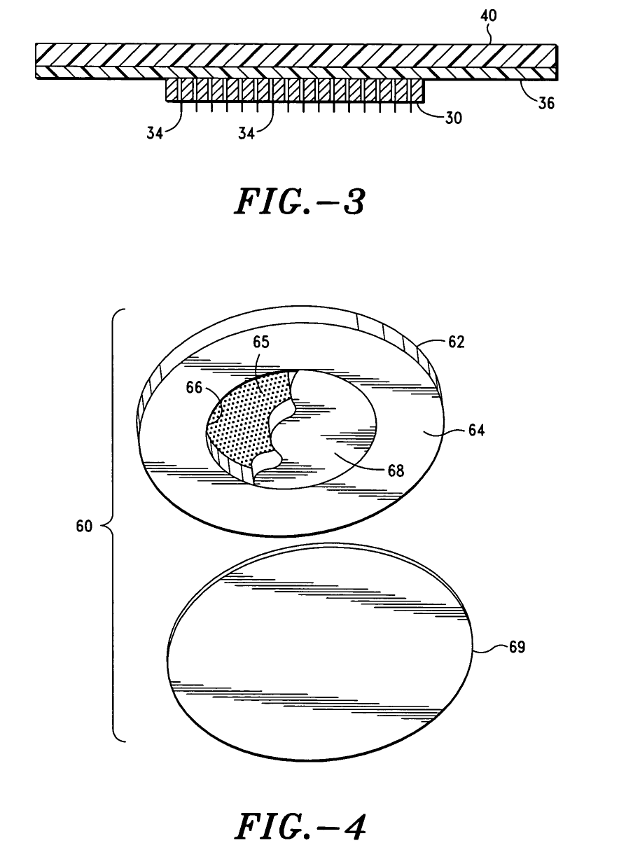

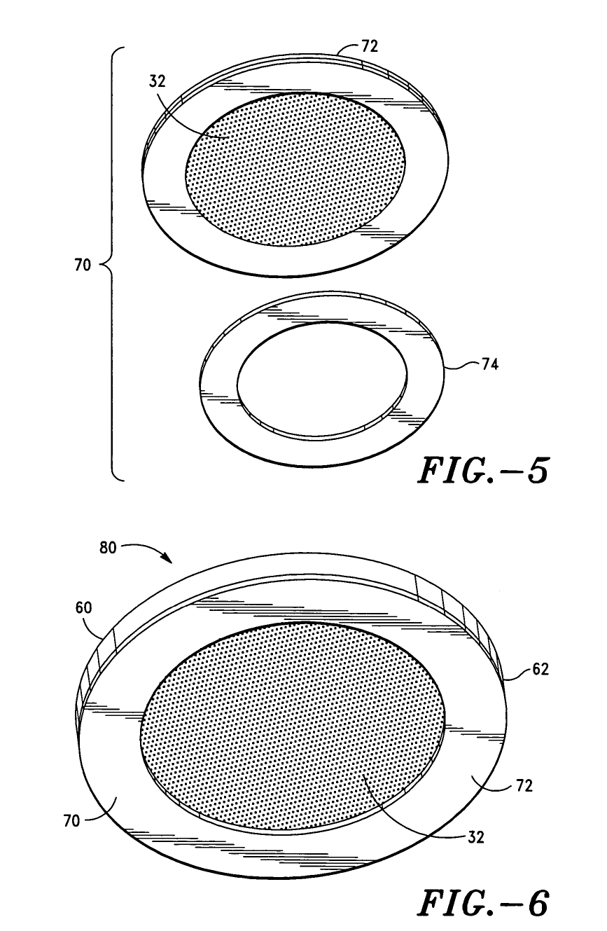

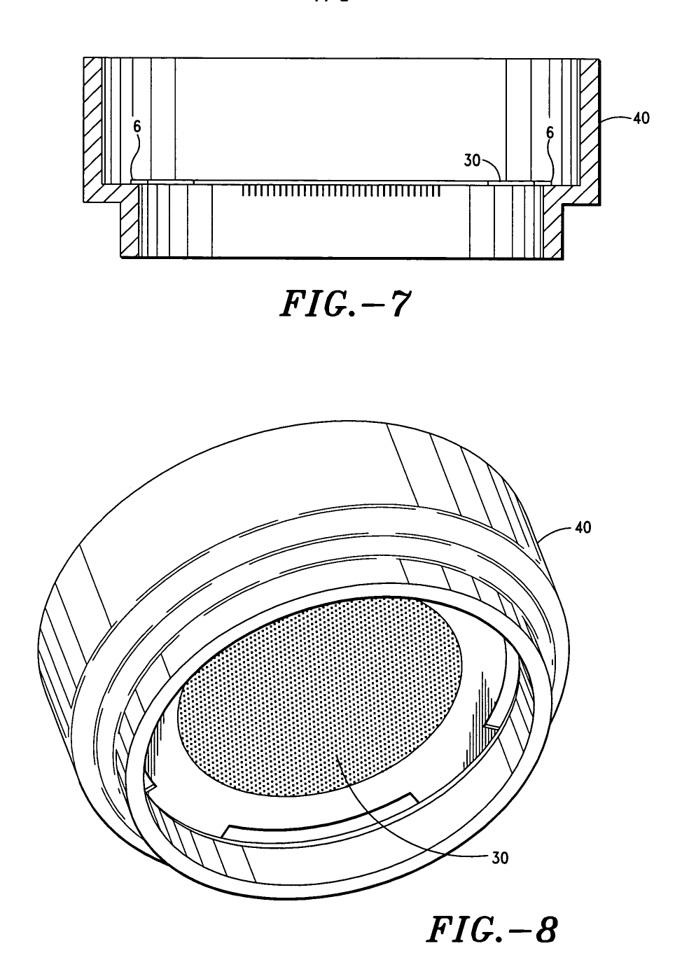

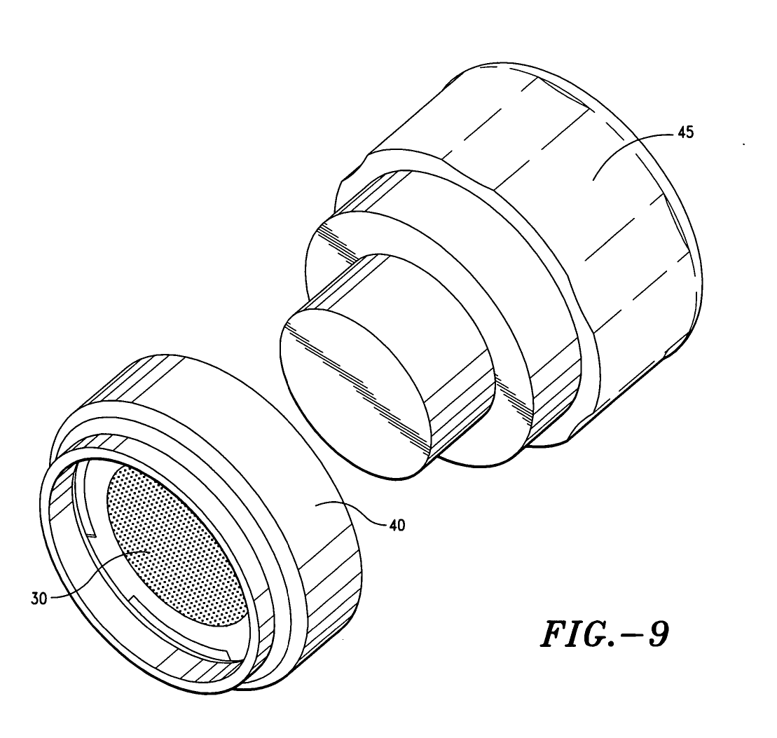

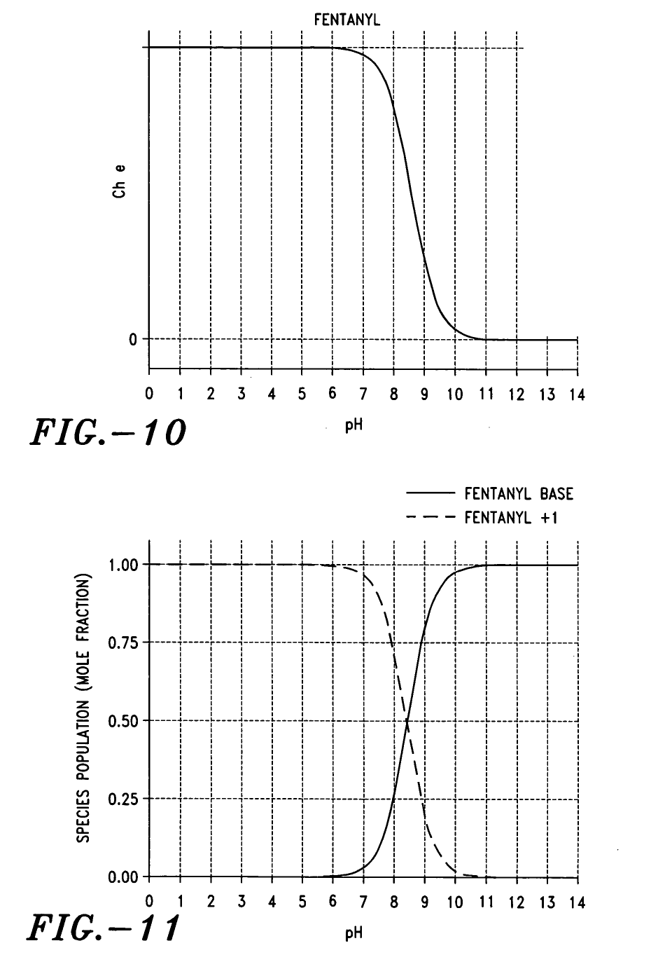

The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 1. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture having at least one fentanyl-based agent that is employed to coat the microprojections and/or arrays thereof. The fentanyl-based agent can be in solution or suspension in the formulation.

The term “biocompatible coating” and “solid coating”, as used herein, is meant to mean and include a “coating formulation” in a substantially solid state.

As indicated above, the present invention generally comprises a delivery system including microprojection member (or system) having a plurality of microprojections (or array thereof) that are adapted to pierce through the stratum corneum into the underlying epidermis layer, or epidermis and dermis layers.

In one embodiment, the microprojections have a biocompatible coating thereon that contains at least one fentanyl-based agent. Upon piercing the stratum corneum layer of the skin, the agent-containing coating is dissolved by body fluid (intracellular fluids and extracellular fluids such as interstitial fluid) and released into the skin (i.e., bolus delivery) for systemic therapy. Preferably, the total dose of fentanyl-based agent delivered transdermally is in the range of approximately 10-1000 μg/day.

According to the invention, the delivery system is particularly suitable for “breakthrough pain” management. For “breakthrough pain” management, the preferred pharmacokinetic profile in humans includes establishment of therapeutically relevant blood levels in less than 30 min, and preferably less than 15 min. In addition, the therapeutically relevant blood levels should be sustained for at least 1 hour and up to 6 hours, preferably, 2-4 hours. In the case of fentanyl, the therapeutically relevant blood levels correspond to at least 0.3 ng/mL.

The delivery system can further be employed for management of chronic pain in patients who require continuous opioid analgesia. For “chronic pain”, the preferred pharmacokinetic profile in humans includes establishment of therapeutically relevant blood levels in less than 2 hours, and preferably less than 1 hour. In addition, the therapeutically relevant blood levels should be sustained for at least 12 hours, and preferably at least 24 hours. In the case of fentanyl, the therapeutically relevant blood levels also correspond to at least 0.3 ng/mL.

Referring now to FIG. 1, there is shown one embodiment of a microprojection member 30 for use with the present invention. As illustrated in FIG. 1, the microprojection member 30 includes a microprojection array 32 having a plurality of microprojections 34. The microprojections 34 preferably extend at substantially a 90° angle from the sheet, which in the noted embodiment includes openings 38.

According to the invention, the sheet 36 can be incorporated into a delivery patch, including a backing 40 for the sheet 36, and can additionally include adhesive 16 for adhering the patch to the skin (see FIG. 3). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², preferably, at least approximately 100 microprojections/cm², and more preferably, in the range of at least approximately 200-3000 microprojections/cm². Preferably, the number of openings per unit area through which the agent passes is at least approximately 10 openings/cm² and less than about 3000 openings/cm².

As indicated, the microprojections 34 preferably have a projection length less than 1000 microns.

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymer. Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. Provisional Application No. 60/484,142, which is incorporated by reference herein.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the fentanyl-base agent to be delivered can be contained in the hydrogel formulation disposed in a gel pack reservoir (discussed in detail below), contained in a biocompatible coating that is disposed on the microprojection member 30 or contained in both the hydrogel formulation and the biocompatible coating.

Referring now to FIG. 2, there is shown a microprojection member 30 having microprojections 34 that include a biocompatible coating 35. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

Referring now to FIGS. 7 and 8, for storage and application, the microprojection member 30 is preferably suspended in a retainer ring 40 by adhesive tabs 6, as described in detail in U.S. application Ser. No. 09/976,762 (Pub. No. 2002/0091357), which is incorporated by reference herein in its entirety.

After placement of the microprojection member 30 in the retainer ring 40, the microprojection member 30 is applied to the patient's skin. Preferably, the microprojection member 30 is applied to the patient's skin using an impact applicator 45, such as shown in FIG. 8 and described in Co-Pending U.S. application Ser. No. 09/976,978, which is incorporated by reference herein in its entirety.

As indicated, according to one embodiment of the invention, the coating formulations applied to the microprojection member 30 to form solid biocompatible coatings can comprise aqueous and non-aqueous formulations having at least one fentanyl-based agent. According to the invention, the fentanyl-based agent can be dissolved within a biocompatible carrier or suspended within the carrier.

In a preferred embodiment, the fentanyl-based agent is selected from the group consisting of fentanyl base, fentanyl salts, including chloride and citrate, simple derivatives of fentanyl and closely related molecules, including, without limitation, remifentanyl, sufentanyl, alfentanyl, lofentanyl and carfentanyl.

Suitable fentanyl salts include, without limitation, acetate, propionate, butyrate, pentanoate, hexanoate, heptanoate, levulinate, chloride, bromide, citrate, succinate, maleate, glycolate gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate, sulfonate, tricarballylicate, malonate, adipate, citraconate, glutarate, itaconate, mesaconate, citramalate, dimethylolpropionate, tiglicate, glycerate, methacrylate, isocrotonate, β-hydroxybutyrate, crotonate, angelate, hydracrylate, ascorbate, aspartate, glutamate.

Suitable simple fentanyl derivatives include, without limitation, alpha-methyl fentanyl, 3-methyl fentanyl and 4-methyl fentanyl.

The noted fentanyl-based agents can be in various forms, such as free bases, acids, charged or uncharged molecules, components of molecular complexes or nonirritating, pharmaceutically acceptable salts.

In one embodiment of the invention, the fentanyl-based agent comprises in the range of approximately 1-30 wt. % of the coating formulation.

Table 1 shows the impact of pH of the agent formulation on the solubility of fentanyl-based agent coatings.

TABLE 1

| | | Solubility | |
|---|---|---|---|
| | Solubility of | of citrate | |
| pH of | HCl salt | salt | |
| solution | (mg/ml) | (mg/ml) | Comments |
| 2.3 | — | 54 | pH of citrate salt was adjusted with |
| | | | 0.1 N HCl |
| 3 | ˜25 | — | pH of HCl salt was adjusted with |
| | | | 0.01 N HCl or 0.99 M NaOH |
| 3.4 | — | 34 | pH of citrate salt was adjusted with |
| | | | 0.1 N HCl |
| 3.1 | — | 32 | Citrate-HCl buffer (pH 2) was used |
| 3.8 | — | 24 | Citrate-HCl buffer (pH 4) was used |
| 4.0 | ˜25 | — | Citrate-NaOH buffer (pH 6) was |
| | | | used |
| 4.6 | — | 45 | pH of HCl salt was adjusted with |
| | | | 0.01 N HCl or 0.99 M NaOH |
| 5.0 | ˜25 | — | Citrate-NaOH buffer (pH 4) was |
| | | | used |
| 5.6 | — | 10 | Citrate buffer (pH 6) |
| 6.0 | ˜25 | — | pH of HCl salt was adjusted with |
| | | | 0.01 N HCl or 0.99 M NaOH |
| 6.6 | ˜5 | 9.1 | pH of HCl salt was adjusted with |
| | | | 0.01 N HCl or 0.99 M NaOH. |
| | | | Citrate buffer (pH 7) used for citrate |
| | | | salt |
| 7.0 | ˜1 | — | pH of HCl salt was adjusted with |
| | | | 0.01 N HCl or 0.99 M NaOH |
| 7.4 | — | 3.1 | Citrate buffer (pH 8) |

Referring now to FIG. 10, there is shown the predicted charge profile of a fentanyl-based agent, a small molecule having one basic pKa with a value of approximately 8.5. Referring now to FIG. 11, there is shown the predicted mole ratios of the net charged species of fentanyl.

As illustrated in FIG. 11, the neutral species only exists in significant amounts above pH 6. Above pH 6, fentanyl is expected to precipitate out of an aqueous solution.

Accordingly, in a preferred embodiment, the pH of the coating formulation is below approximate pH 6. More preferably, the pH of the coating formulation is in the range of approximately pH 2-6. Even more preferably, the pH of the coating formulation is in the range of approximately pH 2-5.5.

In one embodiment of the invention, the coating formulation includes at least one of the aforementioned buffers.

In one embodiment of the invention, the coating formulation includes at least one surfactant. Surfactants exhibit the ability to form micelles and can improve the solubility of solid coatings formed from small molecule agents, such as fentanyl, that otherwise can have poor solublility. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, alkoxylated alcohols such as laureth-4, Triton X-100, Triton X-305, and Brij 35. Most preferred surfactants include Tween 20, Tween 80, and SDS.

In one embodiment of the invention, the concentration of the surfactant is in the range of approximately 0.01-20 wt. % of the coating solution formulation.

In a further embodiment of the invention, the coating formulation includes at least one polymeric material or polymer that has amphiphilic properties. Examples of the noted polymers include, without limitation, cellulose derivatives, such as hydroxyethylcellulose (HEC), hydroxypropylmethylcellulose (HPMC), hydroxylpropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation. Even more preferably, the concentration of the polymer is in the range of approximately 0.1-5 wt. % of the coating formulation.

According to the invention, the coating formulation can further include a hydrophilic polymer. Preferably the hydrophilic polymer is selected from the following group: hydroxyethyl starch, dextran, poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation is preferably in the range of approximately 1.0-30 wt. %, more preferably, in the range of approximately 1-20 wt. % of the coating formulation. Even more preferably, the concentration of the hydrophilic polymer is in the range of approximately 0.1-5 wt. % of the coating formulation.

According to the invention, the coating formulation can further include a biocompatible carrier, such as those disclosed in Co-Pending U.S. application Ser. No. 10/127,108, which is incorporated by reference herein in its entirety. Examples of biocompatible carriers include human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation is preferably in the range of approximately 2-70 wt. %, more preferably, in the range of approximately 5-50 wt. % of the coating formulation.

In a further embodiment, the coating formulation includes at least one stabilizing agent, which can comprise, without limitation, a non-reducing sugar, a polysaccharide or a reducing sugar. Suitable non-reducing sugars for use in the methods and compositions of the invention include, for example, sucrose, trehalose, stachyose, or raffinose. Suitable polysaccharides for use in the methods and compositions of the invention include, for example, dextran, soluble starch, dextrin, and insulin. Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

The coating formulations and, hence, biocompatible coatings of the invention can further include a vasoconstrictor, such as those disclosed in Co-Pending U.S. application Ser. No. 10/674,626, which is incorporated by reference herein in its entirety. As set forth in the noted Co-Pending Application, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the coating formulations and, hence, solid biocompatible coatings of the invention (or the hydrogel formulations or solid film, discussed below) is particularly useful to prevent bleeding that can occur following application of the microprojection member or array and to prolong the pharmacokinetics of the fentanyl-based agent through reduction of the blood flow at the application site and reduction of the absorption rate from the skin site into the system circulation.

The concentration of the vasoconstrictor, if employed, is preferably in the range of approximately 0.1 wt. % to 10 wt. % of the coating formulation.

In yet another embodiment of the invention, the coating formulation includes at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride) and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent. Presently preferred solubilising/complexing agents include alpha-cyclodextrin, beta-cyclodextrin, gamma-cyclodextrin, glucosyl-alpha-cyclodextrin, maltosyl-alpha-cyclodextrin, 2-hydroxypropyl-beta-cyclodextrin, 2-hydroxypropyl-gamma-cyclo-dextrin, hydroxyethyl-beta-cyclodextrin, methyl-beta-cyclodextrin, sulfobutylether-alpha-cyclodextrin, sulfobutylether-beta-cyclodextrin, and sulfobutylether-gamma-cyclodextrin. Most preferred solubilising/complexing agents are beta-cyclodextrin, hydroxypropyl beta-cyclodextrin, 2-hydroxypropyl-beta-cyclodextrin and sulfobutylether7 beta-cyclodextrin.

The concentration of the solubilising/complexing agent, if employed, is preferably in the range of approximately 1 wt. % to 20 wt. % of the coating formulation.

It is well known that cyclodextrins, such as the cyclodextrins disclosed herein, have a hydrophobic ring that can associate with fentanyl's benzene rings to increase solubility. Indeed, it has been established that the addition of hydroxybeta-cyclodextrin in solution will improve the solubility of fentanyl citrate. It has also been established that increasing the pH of hydroxybeta-cyclodextrin can also increase the solubility of fentanyl (see, e.g., C. Holvoet, et al., J. Pharm. 2000, 265, pp. 13-26). Thus, a combination of pH adjustment and the addition of a solubilising/complexing agent is likely to have the greatest impact on solubility.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the range of approximately 1 wt. % to 50 wt. % of the coating formulation.

In yet another embodiment of the invention, the coating formulation includes a suspension agent or carrier, which can form a homogenous mixture solid dispersion with the fentanyl-based agent. The solid dispersion exhibits improved solubility owing to the greater solubility of the carrier. Suitable suspension agents include, without limitation, polyethylene glycol (PEG) and polyvinylpyrrolidine (PVP). A currently preferred suspension agent is PVP (50 kDa).

Other known formulation adjuvants can also be added to the coating formulations provided they do not adversely affect the necessary solubility and viscosity characteristics of the coating formulation and the physical integrity of the dried coating.

Preferably, the coating formulations have a viscosity less than approximately 500 centipoise and greater than 3 centipose.

In one embodiment of the invention, the coating thickness is less than 25 microns, more preferably, less than 10 microns as measured from the microprojection surface.

The desired coating thickness is dependent upon several factors, including the required dosage and, hence, coating thickness necessary to deliver the dosage, the density of the microprojections per unit area of the sheet, the viscosity and concentration of the coating composition and the coating method chosen.

In all cases, after a coating has been applied, the coating formulation is dried onto the microprojections 34 by various means. In a preferred embodiment of the invention, the coated microprojection member 30 is dried in ambient room conditions. However, various temperatures and humidity levels can be used to dry the coating formulation onto the microprojections. Additionally, the coated member can be heated, lyophilized, freeze dried or similar techniques used to remove the water from the coating.

Referring now to FIG. 6, there is shown a further microprojection (or delivery) system (designated generally “80”) that can be employed within the scope of the present invention. As illustrated in FIG. 6, the system 60 includes a gel pack 62 and a microprojection assembly 70, having a microprojection member, such as the microprojection member 30 shown in FIG. 1.

Referring now to FIG. 4, the gel pack 62 includes a housing or ring 64 having a centrally disposed reservoir or opening 66 that is adapted to receive a predetermined amount of a hydrogel formulation 68 therein. As illustrated in FIG. 4, the ring 64 further includes a backing member 65 that is disposed on the outer planar surface of the ring 64. Preferably, the backing member 65 is impermeable to the hydrogel formulation.

In a preferred embodiment, the gel pack 60 further includes a strippable release liner 69 that is adhered to the outer surface of the gel pack ring 64 via a conventional adhesive. As described in detail below, the release liner 69 is removed prior to application of the gel pack 60 to the applied (or engaged) microprojection assembly 70.

Referring now to FIG. 5, the microprojection assembly 70 includes a backing membrane ring 72 and a similar microprojection array 32. The microprojection assembly further includes a skin adhesive ring 74.

Further details of the illustrated gel pack 60 and microprojection assembly 70, as well as additional embodiments thereof that can be employed within the scope of the present invention are set forth in Co-Pending Provisional Application No. 60/514,433, filed Oct. 24, 2003, which is incorporated by reference herein in its entirety.

As indicated above, in at least one embodiment of the invention, the hydrogel formulation contains at least one fentanyl-based agent. In an alternative embodiment of the invention, the hydrogel formulation is devoid of a fentanyl-based agent and, hence, is merely a hydration mechanism.

According to the invention, when the hydrogel formulation is devoid of a fentanyl-based agent, the fentanyl-based agent is either coated on the microprojection array 32, as described above, or contained in a solid film, such as disclosed in PCT Pub. No. WO 98/28037, which is similarly incorporated by reference herein in its entirety, on the skin side of the microprojection array 32, such as disclosed in the noted Co-Pending Application No. 60/514,433 or the top surface of the array 32.

As discussed in detail in the Co-Pending Application, the solid film is typically made by casting a liquid formulation consisting of the biologically active agent, a polymeric material, such as hydroxyethyl starch, dextran, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), or pluronics, a plasticising agent, such as glycerol, propylene glycol, or polyethylene glycol, a surfactant, such as Tween 20 or Tween 80, and a volatile solvent, such as water, isopropanol, or ethanol. Following casting and subsequent evaporation of the solvent, a solid film is produced.

Preferably, the hydrogel formulations of the invention comprise water-based hydrogels. Hydrogels are preferred formulations because of their high water content and biocompatibility.

As is well known in the art, hydrogels are macromolecular polymeric networks that are swollen in water. Examples of suitable polymeric networks include, without limitation, dextran, hydroxyethyl starch, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. These polymers can be obtained in various grades presenting different average molecular weight and therefore exhibit different rheological properties.

Preferably, the concentration of the polymeric material is in the range of approximately 0.5-40 wt. % of the hydrogel formulation.

The hydrogel formulations of the invention preferably have sufficient surface activity to insure that the formulations exhibit adequate wetting characteristics, which are important for establishing optimum contact between the formulation and the microprojection array and skin and, optionally, the solid film.

According to the invention, adequate wetting properties are achieved by incorporating a wetting agent, such as a surfactant or polymeric material having amphiphilic properties, in the hydrogel formulation. Optionally, a wetting agent can also be incorporated in the solid film.

According to the invention, the surfactant(s) can be zwitterionic, amphoteric, cationic, anionic, or nonionic. Examples of suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laureate, and alkoxylated alcohols such as laureth-4. Most preferred surfactants include Tween 20, Tween 80, and SDS.

Examples of suitable polymers include, without limitation, cellulose derivatives, such as hydroxyethyl starch, hydroxyethylcellulose (HEC), hydroxypropyl-methylcellulose (HPMC), hydroxypropycellulose (HPC), methylcellulose (MC), hydroxyethylmethylcellulose (HEMC), or ethylhydroxyethylcellulose (EHEC), as well as pluronics and dextran.

Preferably, the concentration of the surfactant is in the range of approximately 0.001-2 wt. % of the hydrogel formulation. The concentration of the polymer that exhibits amphiphilic properties is preferably in the range of approximately 0.5-40 wt. % of the hydrogel formulation.

As will be appreciated by one having ordinary skill in the art, the noted wetting agents can be used separately or in combinations.

In a further embodiment of the invention, the hydrogel formulation includes a solubilizing/complexing agent, which can comprise alpha-cyclodextrin, beta-cyclodextrin, gamma-cyclodextrin, glucosyl-alpha-cyclodextrin, maltosyl-alpha-cyclodextrin, glucosyl-beta-cyclodextrin, maltosyl-beta-cyclodextrin, hydroxypropyl-beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, and sulfobutylether-gamma-cyclodextrin. Most preferred are beta-cyclodextrin, hydroxypropyl-beta-cyclodextrin, 2-hydroxypropyl-beta-cyclodextrin and sulfobutylether7 beta-cyclodextrin.

In another embodiment of the invention, the hydrogel formulation includes at least one non-aqueous solvent, such as ethanol, isopropanol, methanol, propanol, butanol, propylene glycol, dimethyl sulphoxide and polyethylene glycol 400. Preferably, the non-aqueous solvent is present in the range of approximately 1 wt. % to 50 wt. % of the hydrogel formulation.

According to the invention, the hydrogel formulations can similarly include at least one pathway patency modulator, such as those disclosed in Co-Pending U.S. application Ser. No. 09/950,436. As indicated above, the pathway patentcy modulator can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextran sulfate sodium, and EDTA.

The hydrogel formulation can further include at least one vasoconstrictor. Suitable vasoconstrictors include, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

The hydrogel formulations of the invention exhibit adequate viscosity so that the formulation can be contained in the gel pack 60, keeps its integrity during the application process, and is fluid enough so that it can flow through the microprojection assembly openings and into the skin pathways.

For hydrogel formulations that exhibit Newtonian properties, the viscosity of the hydrogel formulation is preferably in the range of approximately 2-300 Poises (P), as measured at 25° C. For shear-thinning hydrogel formulations, the viscosity, as measured at 25° C., is preferably in the range of 1.5-30.P or 0.5 and 10 P, at shear rates of 667/s and 2667/s, respectively. For dilatant formulations, the viscosity, as measured at 25° C., is preferably in the range of approximately 1.5-30 P, at a shear rate of 667/s.

As indicated, in at least one embodiment of the invention, the hydrogel formulation contains at least one fentanyl-based agent. According to the invention, when the hydrogel formulation contains one of the aforementioned fentanyl-based agents, the fentanyl-based agent can be present at a concentration in excess of saturation or below saturation. The amount of a fentanyl-based agent employed in the microprojection system will be that amount necessary to deliver a therapeutically effective amount of the fentanyl-based agent to achieve the desired result. In practice, this will vary widely depending upon the particular fentanyl-based agent, the site of delivery, the severity of the condition, and the desired therapeutic effect.

In one embodiment of the invention, the concentration of the fentanyl-based agent is in the range of at least 0.1-10 wt. % of the hydrogel formulation.

Preferably, the dose of fentanyl-based agent delivered transdermally is in the range of approximately 10-1000 μg/day.

In accordance with yet another embodiment of the invention, the microprojection system for delivering a fentanyl-based agent comprises (i) a gel pack containing a hydrogel formulation; and (ii) a microprojection member having top and bottom surfaces, a plurality of openings that extend through the microprojection member and a plurality of stratum corneum-piercing microprotrusions that project from the bottom surface of the microprojection member, the microprojection member including a solid film having at least one fentanyl-based agent. Details of the noted system are set forth in Co-Pending Application No. 60/514,433, which is incorporated by reference herein in its entirety.

In accordance with one embodiment of the invention, the solid film is disposed proximate the top surface of the microprojection member. In another embodiment, the solid film is disposed proximate the bottom surface of the microprojection member.

In a preferred embodiment, the hydrogel formulation is devoid of a fentanyl-based agent.

In one embodiment, the solid film is made by casting a liquid formulation consisting of the fentanyl-based agent, a polymeric material, such as hyroxyethyl starch, dextran, hydroxyethylcellulose (HEC), hydroxypropylmethylcellulose (HPMC), hydroxypropycellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone), or pluronics, a plasticising agent, such as glycerol, propylene glycol, or polyethylene glycol, a surfactant, such as Tween 20 or Tween 80, and a volatile solvent, such as water, isopropanol, methanol or ethanol.

In one embodiment, the liquid formulation used to produce the solid film comprises: 0.1-10 wt. % fentanyl-based agent, 5-40 wt. % polymer, 5-40 wt. % plasticiser, 0-2 wt. % surfactant, and the balance of volatile solvent.

Following casting and subsequent evaporation of the solvent, a solid film is produced.

Preferably, the fentanyl-based agent is present in the liquid formulation used to produce the solid film at a concentration in the range of approximately 0.1-10 wt. %.

Preferably, the pH of the liquid formulation used to produce the solid film is below about 6. More preferably, the pH of the formulation used to produce the solid film is in the range of approximately 2-6. Even more preferably, the pH of the liquid formulation used to produce the solid film is in the range of approximately 2-5.5.

In another embodiment, the liquid formulation used to produce the solid film includes a stabilizing agent, which can comprise, without limitation, a non-reducing sugar, a polysaccharide or a reducing sugar. Suitable non-reducing sugars for use in the methods and compositions of the invention include, for example, sucrose, trehalose, stachyose, or raffinose. Suitable polysaccharides for use in the methods and compositions of the invention include, for example, dextran, soluble starch, dextrin, and insulin. Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

In one embodiment of the invention, the liquid formulation used to produce the solid film includes at least one of the aforementioned buffers.

In another embodiment of the invention, the liquid formulation used to produce the solid film includes at least one of the aforementioned complexing/solubilizing agents.

In a further embodiment of the invention, the liquid formulation used to produce the solid film includes at least one of the aforementioned vasoconstrictors.

In accordance with one embodiment of the invention, the method for delivering a fentanyl-based agent contained in a biocompatible coating on the microprojection member comprises the following steps: the coated microprojection member 30 is initially applied to the patient's skin via an actuator, wherein the microprojections 34 pierce the stratum corneum. The coated microprojection member 30 is preferably left on the skin for a period lasting from 5 seconds to 24 hours. Following the desired wearing time, the microprojection member 30 is removed.

Preferably, the dose of fentanyl-based agent delivered transdermally is in the range of approximately 10-1000 μg/day.

In accordance with a further embodiment of the invention, the method for delivering a fentanyl-based agent contained in a solid film disposed proximate (or on) a microprojection member comprises the following steps: the microprojection member 30 is initially applied to the patient's skin via an actuator, wherein the microprojections 34 pierce the stratum corneum. The microprojection member 30 is preferably left on the skin for a period lasting from 5 seconds to 24 hours. Following the desired wearing time, the microprojection member 30 is removed.

Preferably, the dose of fentanyl-based agent delivered transdermally is in the range of approximately 10-1000 μg/day.

In another embodiment of the invention, the microprojection assembly 70 is applied to the patient's skin. After application of the microprojection assembly 70, the release liner 69 is removed from the gel pack 60. The gel pack 60 is then placed on the microprojection assembly 70, whereby the hydrogel formulation 68 is released from the gel pack 60 through the openings 38 in the microprojection array 32, passes through the microslits in the stratum corneum formed by the microprojections 34, migrates down the outer surfaces of the microprojections 34 and through the stratum corneum to achieve local or systemic therapy.

Preferably, the dose of fentanyl-based agent delivered transdermally is in the range of approximately 10-1000 μg/day.

Preferably, the gel pack 60 is left on the patient's skin for a period in the range of approximately 5 min to 7 days. Following the desired wearing time, the gel pack 60 and microprojection assembly 70 are removed from the skin.

In one embodiment of the invention, the microprojection assembly 70 includes a microprojection array 34 having a biocompatible coating disposed thereon that includes at least one fentanyl-based agent, as illustrated in FIG. 2.

In another embodiment, the fentanyl-based agent is contained in a hydrogel formulation in the gel pack 60.

In a further embodiment, the fentanyl-based agent is contained in a hydrogel formulation in the gel pack 60 and in a biocompatible coating applied to the microprojection assembly 70.

According to a further embodiment of the invention, the microprojection assembly 70 is applied to the patient's skin and immediately removed. The release liner 69 is then removed from the gel pack 60 and the gel pack 60 is placed on the pretreated skin, whereby the hydrogel formulation 68 is released from the gel pack 60 and passes through the microslits in the stratum corneum formed by the microprojections 34.

Preferably, the gel pack 60 is left on the patient's skin for a period in the range of approximately 5 min to 7 days. Following the desired wearing time, the gel pack 60 is removed from the skin.

In the noted embodiment, the fentanyl-based agent is contained in the hydrogel formulation in the gel pack 60.

Preferably, the dose of fentanyl-based agent delivered transdermally is in the range of approximately 10-1000 μg/day.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169,383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by attained in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods such as sonophoresis or piezoelectric devices can be used in conjunction with the invention.

When the invention is employed in conjunction with electrotransport, sonophoresis or piezoelectric systems, the microprojection assembly 70 is first applied to the skin as explained above. The release liner 69 is removed from the gel pack 60, which is part of the electrotransport, sonophoresis or piezoelectric system. This assembly is then placed on the skin template, whereby the hydrogel formulation 68 is released from the gel pack 60 and passes through the microslits in the stratum corneum formed by the microprojections 34 to achieve local or systemic therapy with additional facilitation of drug transport via the electrotransport, sonophoresis or piezoelectric processes. When the invention is employed in conjunction with one of the noted systems, the total skin contact area can be in the range of approximately 2-120 cm².

### EXAMPLES

The following examples are given to enable those skilled in the art to more clearly understand and practice the present invention. They should not be considered as limiting the scope of the invention but merely as being illustrated as representative thereof.

### Example 1

An aqueous solution containing 2.5 wt. % fentanyl citrate and having a pH of approximately pH 3.8 is prepared. Sufficient fluorescein is added to the solution to generate a 0.001M concentration. This agent is used to assess the quality of the coating after drying.

A strip of titanium foil is prepared by washing the surface with an alkaline detergent and drying. Five microliters of the coating solution (or formulation) is applied and dried for four hours at room temperature. The quality of the coating is found to be very poor when viewed under a fluorescent microscope, demonstrating poor wetting properties of the fentanyl solution. When 0.1 wt. % hydroxyethyl cellulose (Dow Chemical, Midland Mich.) is added to the same coating solution, the coating is noticeably improved.

### Example 2

A 2.5 wt. % fentanyl citrate solution having a pH of approximately pH 3.8 is prepared in water. To that solution is added 0.1 wt. % hydroxyethyl cellulose (Mₙ=1000 KDa, Mw=1900 KDa) and 0.2 wt. % of the surfactant Tween 20. The coating solution is then applied to the microprojections using the coating methods described in U.S. Publication No. 2002/0132054, which is incorporated by reference herein in its entirety. The coating is evaluated and found to be well distributed across the projections. The coated and dried projections of a 2 cm² device is found to contain 50 micrograms of fentanyl base. When the device is applied in humans using the applicator described in U.S. Publication 2002/0123675 for a duration of 1 hour, delivery of more than 70% of the fentanyl contained on the projections is achieved.

### Example 3

A 1.5 wt. % fentanyl citrate solution having a pH of approximately pH 3.8 is prepared in water. To that solution is added 2 wt. % hydroxyethyl cellulose and 0.2 wt. % of the surfactant Tween 20. The resulting gel is then incorporated in the microprojection reservoir system. The device is applied in humans for 8 h as described in U.S. Application Nos. 60/514,433 and 60/514,387, which are incorporated herein in their entirety. Following application, blood samples are taken at various times and evaluated for fentanyl content. Pharmacokinetic evaluation of the results demonstrate fast onset and prolonged delivery for the duration of the application time.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A system for transdermally delivering a fentanyl-based agent, comprising a microprojection member having a plurality of stratum corneum-piercing microprojections andan agent formulation containing said fentanyl-based agent, said formulation being adapted for transdermal delivery.

2. The system of claim 1, wherein said fentanyl-based agent is selected from the group consisting of fentanyl base, fentanyl salts, alpha-methyl fentanyl, 3-methyl fentanyl, 4-methyl fentanyl, other simple fentanyl derivatives, remifentanyl, sufentanyl, alfentanyl, lofentanyl and carfentanyl.

3. The system of claim 1, wherein said fentanyl-based agent comprises a fentanyl salt formed in conjunction with an ion selected from the group consisting of acetate, propionate, butyrate, pentanoate, hexanoate, heptanoate, levulinate, chloride, bromide, citrate, succinate, maleate, glycolate gluconate, glucuronate, 3-hydroxyisobutrate, 2-hydroxyisobutyrate, lactate, malate, pyruvate, fumarate, tartarate, tartronate, nitrte, phosphate, benzene sulfonate, methane sulfonate, sulfate, sulfonate, tricarballylicate, malonate, adipate, citraconate, glutarate, itaconate, mesaconate, citramalate, dimethylolpropionate, tiglicate, glycerate, methacrylate, isocrotonate, b-hydroxybutyrate, crotonate, angelate, hydracrylate, ascorbate, aspartate and glutamate.

4. The system of claim 1, wherein said agent formulation includes said fentanyl-based agent in the range of approximately 1-60 wt. % of said formulation.

5. The system of claim 4, wherein said agent formulation includes said fentanyl-based agent in the range of approximately 5-30 wt. % of said formulation.

6. The system of claim 1, wherein said agent formulation comprises a biocompatible coating disposed on said microprojection member, said agent formulation being formed from a coating formulation.

7. The system of claim 6, wherein said agent formulation further comprises at least one buffer.

8. The system of claim 7, wherein said buffer is selected from the group consisting of ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

9. The system of claim 7, wherein said coating formulation has a pH in the range of approximately 2-6.

10. The system of claim 9, wherein said coating formulation has a pH in the range of approximately 2-5.5.

11. The system of claim 7, wherein said coating formulation includes a surfactant.

12. The system of claim 11, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, Triton X-100, Triton X-305, Brij 35, polysorbates, such as Tween 20 and Tween 80, sorbitan derivatives, sorbitan laurate, alkoxylated alcohols, and laureth-4.

13. The system of claim 7, wherein said coating formulation includes an amphiphilic polymer.

14. The system of claim 13, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

15. The system of claim 7, wherein said coating formulation includes a hydrophilic polymer.

16. The system of claim 15, wherein said hydrophilic polymer is selected from the group consisting of poly(vinyl alcohol), poly(ethylene oxide), poly(2-hydroxyethylmethacrylate), poly(n-vinyl pyrolidone), polyethylene glycol and mixtures thereof.

17. The system of claim 7, wherein said coating formulation includes a biocompatible carrier.

18. The system of claim 17, wherein said biocompatible polymer is selected from the group consisting of human albumin, bioengineered human albumin, polyglutamic acid, polyaspartic acid, polyhistidine, pentosan polysulfate, polyamino acids, sucrose, trehalose, melezitose, raffinose and stachyose.

19. The system of claim 7, wherein said coating formulation includes a stabilizing agent selected from the group consisting of a non-reducing sugar, a polysaccharide, a reducing sugar, and a DNase inhibitor.

20. The system of claim 19, wherein said stabilizing agent is selected from the group consisting of sucrose, trehalose, stachyose, raffinose, dextran, soluble starch, dextrin, inulin, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, primeverose, vicianose, rutinose, scillabiose, cellobiose, gentiobiose, lactose, lactulose, maltose, melibiose, sophorose, and turanose.

21. The system of claim 7, wherein said coating formulation includes a vasoconstrictor.

22. The system of claim 21, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

23. The system of claim 7, wherein said coating formulation includes a pathway patency modulator.

24. The system of claim 23, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

25. The system of claim 7, wherein said coating formulation includes a solubilising/complexing agent.

26. The system of claim 25, wherein said solubilising/complexing agent is selected from the group consisting of alpha-cyclodextrin, beta-cyclodextrin, gamma-cyclodextrin, glucosyl-alpha-cyclodextrin, maltosyl-alpha-cyclodextrin, glucosyl-beta-cyclodextrin, maltosyl-beta-cyclodextrin, hydroxypropyl beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, sulfobutylether-gamma-cyclodextrin, and sulfobutylether7 beta-cyclodextrin.

27. The system of claim 7, wherein said coating formulation includes at least one non-aqueous solvent.

28. The system of claim 27, wherein said non-aqueous solvent is selected from the group consisting ethanol, isopropanol, methanol, propanol, butanol, propylene glycol, dimethysulfoxide, glycerin, N,N-dimethylformamide and polyethylene glycol 400.

29. The system of claim 7, wherein said coating formulation includes a suspension agent.

30. The system of claim 29, wherein said suspension agent is selected from the group consisting of polyethylene glycol and polyvinylpyrrolidine.

31. The system of claim 7, wherein said coating formulation has a viscosity less than approximately 500 centipoise and greater than 3 centipoise.

32. The system of claim 7, wherein said coating has a thickness less than approximately 25 microns.

33. The system of claim 1, wherein said microprojection member has a microprojection density of at least approximately 100 microprojections/cm².

34. The system of claim 33, wherein said microprojection member has a microprojection density in the range of approximately 200-3000 microprojections/cm².

35. The system of claim 1, wherein each of said microprojections has a length in the range of approximately 50-145 microns.

36. The system of claim 35, wherein each of said microprojections has a length in the range of approximately 70-140 microns.

37. The system of claim 1, further comprising a gel pack, wherein said agent formulation comprises a hydrogel formulation and wherein said gel pack is adapted to receive said hydrogel.

38. The system of claim 37, wherein said fentanyl-based agent comprises in the range of approximately 0.1-10 wt. % of the hydrogel formulation.

39. The system of claim 37, wherein said hydrogel formulation has a pH in the range of approximately 2-6.

40. The system of claim 39, wherein said hydrogel formulation has a pH in the range of approximately 2-5.5.

41. The system of claim 37, wherein said hydrogel formulation includes at least one buffer selected from the group consisting of ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

42. The system of claim 37, wherein said hydrogel comprises a macromolecular polymeric network.

43. The system of claim 42, wherein said macromolecular polymeric network is selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

44. The system of claim 37, wherein said hydrogel formulation includes a surfactant selected from the group consisting of zwitterionic, amphoteric, cationic, anionic, and nonionic.

45. The system of claim 44, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, Tween 20, Tween 80, sorbitan derivatives, sorbitan laurate, alkoxylated alcohols, and laureth-4.

46. The system of claim 37, wherein said hydrogel formulation includes an amphiphilic polymer.

47. The system of claim 46, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

48. The system of claim 37, wherein said hydrogel formulation includes a solubilising/complexing agent.

49. The system of claim 48, wherein said solubilising/complexing agent is selected from the group consisting of alpha-cyclodextrin, beta-cyclodextrin, gamma-cyclodextrin, glucosyl-alpha-cyclodextrin, maltosyl-alpha-cyclodextrin, glucosyl-beta-cyclodextrin, maltosyl-beta-cyclodextrin, hydroxypropyl beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, sulfobutylether-gamma-cyclodextrin, and sulfobutylether7 beta-cyclodextrin.

50. The system of claim 37, wherein said hydrogel formulation includes a pathway patency modulator.

51. The system of claim 50, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

52. The system of claim 37, wherein said hydrogel formulation includes a vasoconstrictor.

53. The system of claim 52, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

54. The system of claim 1, further comprising a solid film formed from a liquid formulation of said agent formulation and a gel pack having a hydrogel formulation.

55. The system of claim 54, wherein said solid film is disposed proximate a top surface of said microprojection member.

56. The system of claim 54, wherein said solid film is disposed proximate a bottom surface of said microprojection member.

57. The system of claim 54, wherein said hydrogel is substantially devoid of said fentanyl-based agent.

58. The system of claim 54, wherein said solid film is formed from said fentanyl-based agent, a polymeric material a plasticising agent, asurfactant and a volatile solvent.

59. The system of claim 58, wherein said liquid formulation comprises 0.1-10 wt. % said fentanyl-based agent, 5-40 wt. % said polymer, 5-40 wt. % said plasticiser, 0-2 wt. % said surfactant, and a balance of said volatile solvent.

60. The system of claim 54, wherein said fentanyl-based agent comprises in the range of approximately 0.1-10 wt. % of said liquid formulation.

61. The system of claim 54, wherein said liquid formulation has a pH in the range of approximately 2-6.

62. The system of claim 61, wherein said liquid formulation has a pH in the range of approximately 2-5.5.

63. The system of claim 54, wherein said liquid formulation includes at least one buffer selected from the group consisting of ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

64. The system of claim 54, wherein said liquid formulation includes a solubilising/complexing agent.

65. The system of claim 64, wherein said solubilising/complexing agent is selected from the group consisting of alpha-cyclodextrin, beta-cyclodextrin, gamma-cyclodextrin, glucosyl-alpha-cyclodextrin, maltosyl-alpha-cyclodextrin, glucosyl-beta-cyclodextrin, maltosyl-beta-cyclodextrin, hydroxypropyl beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, sulfobutylether-gamma-cyclodextrin, and sulfobutylether7 beta-cyclodextrin.

66. The system of claim 54, wherein said liquid formulation includes a pathway patency modulator.

67. The system of claim 66, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

68. The system of claim 54, wherein said liquid formulation includes a vasoconstrictor.

69. The system of claim 68, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

70. A method for transdermally delivering a fentanyl-based agent comprising the steps of:
providing a microprojection member having a plurality of stratum corneum-piercing microprojections having a biocompatible coating of an agent formulation containing said fentanyl-based agent; and
applying said coated microprojection member to a patient's skin via an actuator, wherein said microprojections pierce the stratum corneum and deliver said fentanyl-based agent.

71. The method of claim 70, further comprising the step of maintaining said coated microprojection member is on said skin for a period in the range of approximately 5 seconds to 24 hours.

72. A method for transdermally delivering a fentanyl-based agent, comprising the steps of:
providing a microprojection member with a plurality of stratum corneum-piercing microprojections and a solid film containing said fentanyl-based agent; and
applying said microprojection member to a patient's skin via an actuator.

73. The method of claim 72, further comprising the steps of:
applying a gel pack having a hydrogel formulation substantially devoid of said fentanyl-based agent;
hydrating said solid film with said hydrogel formulation to deliver said fentanyl-based agent.

74. The method of claim 72, further comprising the step of maintaining said microprojection member with said solid film on said skin for a period in the range of approximately 5 seconds to 24 hours.

75. A method for transdermally delivering a fentanyl-based agent, comprising the steps of:
providing a microprojection member with a plurality of stratum corneum-piercing microprojections and a gel pack having a fentanyl-based agent-containing hydrogel formulation;
applying said microprojection member to a patient's skin so that said microprojections form microslits in the stratum corneum; and
placing said gel pack on said microprojection member so that said hydrogel formulation migrates into and through said microslits to deliver said fentanyl-based agent.

76. The method of claim 75, further comprising the step of maintaining said microprojection member and said gel pack on said skin for a period in the range of approximately 5 minutes to 7 days.

77. A method for transdermally delivering a fentanyl-based agent, comprising the steps of:
providing a microprojection member with a plurality of stratum corneum-piercing microprojections;
applying said microprojection member to a patient's skin so that said microprojections form microslits in the stratum corneum;
removing said microprojection member; and
applying a gel pack having a fentanyl-based agent-containing hydrogel formulation to said patient's skin with said microslits to deliver said fentanyl-based agent.

78. The method of claim 77, further comprising the step of maintaining said gel pack on said skin for a period in the range of approximately 5 minutes to 7 days.

79. A method for transdermally delivering a fentanyl-based agent, comprising the steps of:
providing a microprojection member with a plurality of stratum corneum-piercing microprojections having a biocompatible coating containing said fentanyl-based agent;
applying said microprojection member to a patient's skin to form microslits in the stratum corneum and deliver said fentanyl-based agent; and
placing a gel pack having a fentanyl-based agent-containing hydrogel formulation on said microprojection member to deliver said fentanyl-based agent through said microslits.

80. The method of claim 79, further comprising the step of maintaining said microprojection member and said gel pack on said skin for a period in the range of approximately 1 to 6 hours.

81. The method of claim 79, further comprising the step of maintaining said microprojection member and said gel pack on said skin for a period in the range of approximately 2 to 4 hours.
